(19) United States
(12) Patent Application Publication
(10) Pub. No.: US 2010/0119581 A1
(43) Pub. Date: May 13, 2010

(54) Medical Products That Release Pharmaceutically Active Substances

(75) Inventors: Matthias Gratz, Erlangen (DE); Alexander Borck, Aurachtal (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/613,487

(22) Filed: Nov. 5, 2009

(30) Foreign Application Priority Data
Nov. 13, 2008 (DE) 10 2008 043 724.7

(51) Int. Cl. A61K 9/00; A61K 31/337

(52) U.S. Cl. 424/426; 514/449

(57) ABSTRACT

The present invention concerns medical products that release pharmaceutically active substances, the efficiency of which is increased as the result of a combination with an inhibitor of the transport protein P-glycoprotein.

### CROSS REFERENCE TO RELATED APPLICATIONS

This invention claims benefit of priority to Germany patent application serial number DE 10 2008 043 724.7, filed on Nov. 13, 2008; the contents of which is herein incorporated by reference in its entirety.

### FIELD OF THE INVENTION

The present invention concerns medical products that release pharmaceutically, active substances, the efficiency of which is increased as the result of a combination with an inhibitor of the transport protein P-glycoprotein.

### BACKGROUND OF THE INVENTION

One of the most frequent causes of death in the developed world is due to cardiovascular diseases, whereby coronary diseases are of the highest significance. For the treatment of these diseases, intravascular prostheses such as, for example, balloons or stents are introduced into the affected blood vessel of a patient, and if necessary implanted, in order to widen such and to keep it open.

However, because of the intravascular intervention, this can lead to an increased formation of a thrombosis as well as an increased proliferation of smooth muscle cells, which can lead to a renewed closing of the blood vessel (restenosis). Excessive proliferation of scar tissue leads to a restenosis after a longer period of time for approximately 30-40% of all uncoated stents.

In order to prevent the risk factors of a restenosis, many coatings were developed for stents that are intended to offer increased hemo-compatibility. For example, anticoagulating, antimicrobial, anti-inflammatory and antiproliferative agents have been used by themselves or in combination in the coating of stents for a long time. These substances are intended to be released from the coating material of the stent in such a way that they prevent inflammation of the surrounding tissue, overshooting growth of the smooth muscle cells or the clotting of blood.

However, many of these coated stents have the disadvantage that the respective active substances must be used at an increased concentration because of manifestations of resistance by endogenous structures in the body, which can lead to a local intoxication.

These manifestations of resistance, also called multidrug-resistance (MDR), are caused by various membrane-bound transport proteins, which by expending energy attain the ability to remove substances directly from the membrane. The essential characteristic of the transport-based resistance mechanisms consists of a decrease of the intracellular concentration of active substance.

One of the most significant factors of multi-drug resistance is the transport protein P-glycoprotein. P-glycoprotein is of particular significance because it is in a position to recognize many of the compounds that belong to various structural classes and to transport such out of the intracellular space.

Since the discovery of these transport proteins that are responsible for MDR, substantial efforts have been made to analyze active substances for their P-glycoprotein-selective, inhibitory properties and to introduce them into coated, medical devices. As a result of the blocking of P-glycoproteins, the intracellular accumulation of the active substance can be increased in this manner and thereby, the multidrug resistance can be decreased.

Thus, among other things, in DE 600 28 747 T, DE 10 2004 020 856 A, DE 600 26 513 T and DE 601 21 992 T, diverse medical devices are revealed, among others stents, which can be provided with a polymer coating containing a combination of various medications, among others, inhibitors of the transport protein P-glycoprotein. The polymer coating can consist of a number of different polymers.

However, the problem continues to exist that many of the inhibitors of the transport protein P-glycoprotein that are used have an affinity that is too low with respect to P-glycoprotein, so that for an sufficient, intracellular concentration of active substance, the active substances must continue to be used at an increased concentration in the respective coatings.

These increased concentrations of active substance can potentially lead to undesired side reactions in the surrounding cell material and tissue material.

### SUMMARY OF THE INVENTION

The present invention recognizes the problem of making medical products available that make it possible, that as a result of combining a high affinity inhibitor of the transport protein P-glycoprotein with other pharmaceutically active substances, the efficiency of the active substance accumulation in the intracellular space is increased and the use of smaller concentrations of the respective components is made possible to avoid an intoxication of the cell material and the tissue material.

In accordance with the invention, this problem is solved thereby, that a medical product is made available that is entirely or partially coated with a biostable and/or biodegradable polymer layer, which in and/or on the biostable and/or biodegradable polymer layer has at least one inhibitor of the transport protein P-glycoprotein, as well as at least one additional pharmaceutical substance, whereby the at least one inhibitor of the transport protein P-glycoprotein is selected from the group consisting of valspodar (PSC833), elacridar (GF120918), tariquidar (XR9576), zosuquidar (LY335979) and ONT-093 (OC144-093).

### DETAILED DESCRIPTION OF THE INVENTION

In the following, the term inhibitor is used as the equivalent for an inhibitor of the transport protein P-glycoprotein.

Surprisingly, it has been shown that by introducing and/or applying at least one inhibitor of the transport protein P-glycoprotein selected from the group consisting of PSC833, GF120918, XR9576, LY335979 and OC144-093 in and/or on the biostable and/or biodegradable polymer layer, for one, the concentration of the inhibitor and for another, the concentration of the at least one pharmaceutically active substance can be decreased as well. Because of the high affinity of PSC833, GF120918, XR9576, LY335979 and OC144-093 with respect to the P-glycoprotein, even at lower concentrations of the inhibitor the effect of the P-glycoprotein is blocked more strongly, so that a lower level of transport of the active substances takes place that is directed out of the cell. Thus, the efficiency of the medical products according to the invention that release pharmaceutical substances, can be increased significantly.

Medical products within the protective scope of the present invention include any medical devices that are used, at least in part, in order to be placed into the body of the patient. Examples are, implantable devices [such as] cardiac pacemakers, catheters, needle injection catheters, blood clotting filters, vascular transplants, balloons, stent transplants, gall stents, intestinal stents, bronchial lung stents, esophageal stents, ureter stents, aneurism-filling spools and other spool devices, trans-myocardial revascularization devices, percutaneous myocardial revascularization devices. Further, any natural and/or artificial medical products can be used, for example, prostheses, organs, vessels, aortas, heart valves, tubes, organ replacement parts, implants, fibers, hollow fibers, membranes, blood stock, blood containers, titer plates, adsorbing media, dialysators, connection pieces, sensors, valves, endoscopes, filter, pump chambers, as well as other medical products that are intended to have hemo-compatible properties. The term medical products is to be understood broadly and describes especially those products that come in contact with blood for a short time (for example, endoscope) or permanently (for example, stents).

Particularly preferred medical products are balloon catheters and stents. Stents of conventional construction have a filigree support structure of metallic rods, which, for insertion into the body are first present in a non-expanded condition, and which are then expand at the site of the application into an expanded condition. The stent can be coated before or after being crimped onto a balloon.

Preferably, the basic body of the stent consists of a metallic material of one or more metals from the group of iron, magnesium, nickel, wolfram, titanium, zirconium, niobium tantalum, zinc or silicone and perhaps of a second component of one or more metals from the group of lithium, sodium, potassium, calcium, manganese, iron or wolfram, preferably of a zinc-calcium alloy.

In a further example of an embodiment, the basic body consists of a form memory material of one or more materials from the group consisting of nickel-titanium alloys and copper-zinc-aluminum alloys, but preferably of nitinol.

In a further preferred example of an embodiment, the basic body of the stent consists of stainless steel, preferably of a Cr—Ni—Fe steel—here, preferably the alloy 316L—or a Co—Cr steel. Further, the basic body of the stent can consist, at least in part, of plastic and/or ceramic.

In a further example of an embodiment, the basic body of the stent consists of a biocorrodible metallic substance, for example, a biocorrodible alloy selected from the group of magnesium, iron and wolfram; especially, the biocorrodible metallic substance is a magnesium alloy.

A biocorrodible magnesium alloy is to be understood as a metallic structure, the main component of which is magnesium. The main component is the alloy component that has the highest proportion of weight in the alloy. The share of the main component preferably amounts to more than 50% by weight, particularly more than 70%. Preferably, the biocorrodible magnesium alloy contains yttrium and other rare earth elements, as an alloy of this type distinguishes itself because of its physico-chemical properties and high biocompatibility, especially also its degradation products. Particularly preferred is a magnesium alloy of the composition, rare earth elements 5.2-9.9% by weight, thereof yttrium 3.7-5.5% by weight and the remainder <1% by weight, whereby magnesium makes up the missing part of the alloy that competes 100% by weight. In the first clinical tests, this magnesium alloy already confirmed its special suitability in experiments, i.e. it shows high biocompatibility, favorable processing properties, good mechanical values and adequate corrosion behavior for the purposes of use. The collective term “rare earth elements” is understood to mean in the present case, scandium (21), yttrium (39), lanthan (57) and the 14 elements following lanthan (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

In a further example of an embodiment, the stent consists of natural polymers such as, for example, collagen, chitin, chitosan [and] heparin.

The surface of the medical product in accordance with the invention has a complete or partial biostable and/or biodegradable polymer layer that contains at least one inhibitor of the transport protein P-glycoprotein selected from the group consisting of PSC833, GF120918, XR9576, LY335979 and OC144-093, as well as at least one additional pharmaceutically active substance.

The term coating or polymeric carrier matrix is used as synonym for the biostable and/or biodegradable polymer layer within the scope of the present invention.

A biostable and/or biodegradable polymer layer within the scope of the invention is an application at least in sections of the components of the coating onto the medical product. Preferably, the entire surface of the medical product is covered by the coating. The thickness of the layer is preferably in the range of 2 μm to 60 μm, particularly preferred, 10 μm to 30 μm. The medical products in accordance with the invention distinguish themselves, inter alia, thereby, that as a result of the reduction of the active substance—at the same effectiveness—significantly thinner layers can be realized on the medical product. Compared to that, coatings loaded with active substances of conventional medical products are at a size magnitude of 100 μm.

The weight proportion of a polymeric carrier matrix in accordance with the invention of the components that make up the coating preferably amounts to at least 40%, especially preferred at least 70%. The weight proportion of the at least one inhibitor of the transport protein P-glycoprotein of the components forming the coating is preferably not more than 30%, especially preferred, not more than 15%. The weight proportion of the at least one additional pharmaceutically active substance of the components forming the coating is preferably not more than 30%, especially preferred, not more than 15%.

The coating can be applied directly to the medical product. The administration can occur according to the standard procedure for coating. Single layer, but also multi-layer systems (for example, so-called base coat layers, drug coat or top coat layers) can be created. The coating can be applied directly to the basic body of the implant or additional layers can be provided between, for example, for adhesion.

Alternatively, the biostable and/or biodegradable polymer layer containing at least one inhibitor of the transport protein P-glycoprotein selected from the group consisting of PSC833, GF120918, XR9576, LY335979 and OC144-093 as well as at least one additional pharmaceutically active substance can be present as cavity filling or as component of a cavity filling. The medical product, particularly the stent has one or more cavities for this purpose. Cavities are, for example, at the surface of the medical product and can be created, for example, by laser ablation in nano dimensions up to micro meter dimensions. In medical products, particularly stents with a biodegradable basic body, a cavity can also be located in the interior of the basic body, so that the release of the material takes place only after exposure. A person skilled in the art can find an orientation with respect to the design of the cavity in systems that are described in prior art. The term “cavity” thereby comprises, for example, holes and recesses.

The biostable and/or biodegradable polymer layer within the scope of the present invention consists of polymers selected from the group consisting of non-resorbable, permanent polymers and/or resorbable biodegradable polymers.

Particularly preferred, the biostable and/or biodegradable polymer layer consists of polymers selected from the group of polyolefins, polyether ketones, polyether, polyvinyl alcohols, polyvinyl halogenides, polyvinyl esters, polyacrylates, polyhalogene olefins, polyamides, polyamide imides, polysulfons, polyester, polyurethane, silicone, polyphosphazenes, polyphenylene, polymer foams (of styrol and carbonates), polydioxanone, polyglycolide, polylactide, poly-c-caprolactone, ethylvinyl acetate, polyethylene oxide, polyphosphoryl choline, polyhydroxy butyric acids, lipids, polysaccharides, proteins, polypeptides, as well as copolymers, blends and derivatives of these compounds.

Very particularly preferred, the biostable and/or biodegradable polymer layer consists of polymers selected from the group consisting of polypropylene, polyethylene, poly-isobutylene, polybutylene, polyether ether-ketone, polyethylene glycol, polypropylene glycol, polyvinyl alcohols, polyvinyl chloride, polyvinyl fluoride, polyvinyl acetate, polyethyl acrylate, polymethyl acrylate, polytetrafluoro ethylene, polychlortrifluoro ethylene, PA 11, PA 12, PA 46, PA 66, polyamide imide, polyether sulfon, polyphenyl sulfon, polycarbonate, polybutylene terephthalat, polyethylene terephthalat, elastane, pellethane, silicone, polyphosphazene, polyphenylene, polymer foams (of styrols and carbonates), polydioxanone, polyglycolide, poly-l-, poly-d-, and poly-d,l-lactide, as well as poly-ε-caprolactone, ethylvinyl acetate, polyethylene oxide, polyphosphoryl choline, polyhydroxy valerate, cholesterol, cholesterol ester, alginate, chitosan, levan, hyaluronic acid, uronide, heparin, dextran, cellulose, fibrin, albumin, polypeptide and copolymers, blends and derivatives of these compounds.

The biostable and/or biodegradable polymer layer preferably depends on the desired elution speed, as well as on the individual characteristics of the various active substances that are used and on the various resorption or degradation speeds at the site at which the medical product is active.

Within the scope of the present invention, an inhibitor is to be understood as being a blocking substance, i.e. a substance that influences one or more reactions—of chemical, biological or physiological nature—in such a way that they are slowed down, blocked or prevented.

The at least one inhibitor of the transport protein P-glycoprotein selected from the group consisting of PSC833, GF120918, XR9576, LY335979 and OC144-093 is preferably introduced in a concentration between 0.25 to 7 μg/mm² of stent surface, particularly preferred between 0.6 to 3.8 μg/mm² of stent surface into and/or onto the biostable and/or biodegradable polymer layer.

The at least one pharmaceutically active substance is a substance that must be discharged to the environment of the implant in which the medical product, particularly the balloon catheter or a stent is introduced, but it is preferred, that it is released at a very low rate into the blood circulation. The pharmaceutically active substance is preferably selected from the following classes of medications: antimicrobial, antimitotic, antimyotic, antineoplastic, antiphlogistic, antiproliferative, antithrombotic and vasodialatory agents.

Particularly preferred pharmaceutically active substances are triclosan, cephalosporin, amino glycoside, nitrofurantoin, penicillins such as dicloxacillin, oxacillin as well as sulfonamides, metronidazol, 5-fluoruracil, cisplatin, vinblastin, vincristine, epothilones, endostatin, verapamil, statins such as ccrivastatin, atorvastatin, simvastatin, fluvastatin, rosuvastatin as well as lovastatin, angiostatin, angiopeptin, taxanes such as paclitaxel, immuno suppressives or immuno modulators such as, for example, rapamycin or its derivatives such as biolimus, everolimus, deforloimus, novolimus, methotrexate, colchicine, flavopiridol, suramin, cyclosporin A, clotrimazol, flucytosin, griseofulvin, ketoconazol, miconazol, nystatin, terbinafin, steroids such as dexamethasone, prednisolone, corticosterone, budesonid, estrogen, hydrocortisone as well as mesalamine, sulfasalazine, heparin and its derivatives, urokinase, PPack, argatroban, aspirin, abciximab, synthetic antithrombin, bivalirudin, enoxoparin, hirudin, r-hirudin, protamine, prourokinase, streptokinase, warfarin, flavonoids such as 7,3′,4′-trimethoxytlavon as well as dipyramidol, trapidil as well as nitroprusside.

The pharmaceutically active substances individually or in combination are used in the same or in different concentrations.

Particularly preferred is a combination of several antiproliferative active substances. Especially preferred, the medical product that is entirely or partially coated with a biostable and/or biodegradable polymer layer, is provided in or on the biostable and/or biodegradable polymer layer with at least one inhibitor of the transport protein P-glycoprotein selected from the group consisting of PSC833, GF120918, XR9576, LY335979 and OC144-093 as well as at least one pharmaceutically active substance paclitaxel and rapamycin, individually or in combination.

Additionally preferred are combinations of antiproliferatively acting substances with vasodilatory or pleiotropic active substances. Verapamil as well as statins are among the substances that act in this way. Especially preferred, the medical product that is coated with a biostable and/or biodegradable polymer layer is provided in and/or on the biostable and/or biodegradable polymer layer with at least one of the inhibitors of the transport protein P-glycoprotein selected from the group consisting of PSC833, GF120918, XR9576, LY335979, OC144-093, at least one pharmaceutically active substance from the group paclitaxel and rapamycine, as well as an additional pharmaceutically active substance selected from the group consisting of verapamil, atorvastatin, simvastatin and lovastatin, individually or in combination.

The pharmaceutically active substance is preferably contained in a pharmaceutically active concentration of 0.2 to 3.5 μg/mm² of stent surface, further preferred of 0.25 to 0.75 μg/mm² of stent surface.

The medical product according to the invention can have additional inner or outer coatings. An additional outer layer that contains the coating or the cavity filling of at least one inhibitor of the transport protein P-glycoprotein, as well as at least one additional pharmaceutically active substance can provide an entire or partial cover. This outer coating can contain a degrading polymer or consist of such, in particular a polymer from the class of PLGAs (poly(lactic-co-glycolic acid)) or that of the PLGA-PEG block copolymers. If appropriate, in this additional outer layer, an additional active substance can be embedded, which can freely elute or is released during the degradation of the outer coating.

Processes for producing a medical product that releases pharmaceutically active substances are known to one skilled in the art. For example, a process can have the following steps: preparation of a medical product, particularly a stent or balloon catheter; application of a biostable and/or biodegradable polymer layer; and applying onto and/or including at least one inhibitor of the transport protein P-glycoprotein, as well as at least one pharmaceutically active substance applied to and/or included in the biostable and/or biodegradable polymer layer.

An additional aspect of the invention is the use of PSC833, GF120918, XR9576, LY335979 and OC144-093 as inhibitors of the transport protein P-glycoprotein for the production of a medical product that is entirely or partially coated with a biostable and/or biodegradable polymer layer, especially balloon catheters or stents, whereby in and/or on the biostable and/or biodegradable polymer layer at least one inhibitor of the transport protein P-glycoprotein selected from the group consisting of PSC833, GF120918, XR9576, LY335979 and OC144-093 as well as at least one pharmaceutically active substance is contained.

### EXAMPLES

In the following, the invention is explained in more detail using the examples of embodiments, but this is not intended to limit the subject matter of the invention.

### Example of an Embodiment 1

### Coating of a Stent with Equimolar Quantities of PSC833 and Paclitaxel (Ptx)

15.8 mg (0.013 mmol) PSC833 and 11 mg (0.013 mmol) Ptx are dissolved in 100 ml chloroform together with 100 mg poly-l-lactide (PLLA; L210 from Boehringer, Ingelheim) at room temperature. As solubilizer, 5% methanol is added to the solvent. The solution synthesized in this manner is applied to the stent in an immersion process. The surface load of Ptx is 0.5 μg/mm². The quantity of active substance consisting of Ptx and PSC833 is 21% with respect to the polymer (PLLA).

### Example of an Embodiment 2

### Coating of a Stent with 1.2 Equivalent PSC833 and 1 Equivalent Paclitaxel (Ptx)

18.95 mg (0.0156 mmol) PSC833 and 11 mg (0.013 mmol) Ptx are dissolved in 100 ml chloroform together with 100 mg poly-l-lactide (PLLA; L210 from Boehringer, Ingelheim) at room temperature. As solubilizer, 5% methanol is added to the solvent. The solution synthesized in this manner is applied to the stent in an immersion process. The surface load of Ptx is 0.3 μg/mm². The quantity of active substance consisting of Ptx and PSC833 is 23% with respect to the Polymer (PLLA).

### Example of an Embodiment 3

### Coating of a Stent with Equimolar Quantities of GF120918 and Paclitaxel (Ptx)

7.32 mg (0.013 mmol) GF120918 and 11 mg (0.013 mmol) Ptx are dissolved in 100 ml chloroform together with 100 mg poly-l-lactide (PLLA; L210 from Boehringer, Ingelheim) at room temperature. As solubilizer, 5% methanol is added to the solvent. The solution synthesized in this manner is applied to the stent in an immersion process. The surface load of Ptx is 0.5 μg/mm². The quantity of active substance consisting of Ptx and GF120918 is 15% with respect to the polymer (PLLA).

### Example of an Embodiment 4

### Coating of a Stent with 1.2 Equivalent GF120918 and 1 Equivalent Paclitaxel (Ptx)

8.79 mg (0.0156 mmol) GF120918 and 11 mg (0.013 mmol) Ptx are dissolved in 100 ml chloroform together with 100 mg poly-l-lactide (PLLA; L210 from Boehringer, Ingelheim) at room temperature. As salubilizer, 5% methanol is added to the solvent. The solution that is synthesized in this way is applied to the stent in an immersion process. The surface load of Ptx is 0.3 μg/mm². The quantity of active substance consisting of Ptx and GF120918 is 16.5% with respect to the polymer (PLLA).

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A medical product, whereby the surface of the medical product is coated entirely or partially with a biostable and/or biodegradable polymer layer and in and/or on the biostable and/or biodegradable polymer layer there is at least one inhibitor of the transport protein P-glycoprotein, as well as at least one pharmaceutically active substance, wherein the at least one inhibitor of the transport protein P-glycoprotein is selected from the group consisting of PSC833, GF120918, XR9576, LY335979 and OC144-093.

2. The medical product according to claim 1, wherein the biostable and/or biodegradable polymer layer is composed of polymers selected from the group consisting of polyolefins such as polypropylene, polyethylene, poly-isobutylene and polybutylene, as well as polyether ketone such as polyether ether-ketone, as well as polyether such as polyethylene glycol and polypropylene glycol, as well as polyvinyl alcohols, polyvinyl halogenides such as polyvinyl chloride and polyvinyl fluoride, as well as polyvinyl ester such as polyvinyl acetate, polyacrylate, polyethyl acrylate, polymethyl acrylate and polymethylmet acrylate, as well as polyhalogen olefins such as polytetrafluoro ethylene and polychlortrifluoro ethylene, as well as polyamide such as PA 11, PA 12, PA 46 and PA 66, as well as polyamide imide, polysulfons such as polyether sulfon and polyphenyl sulfon, as well as polyester such as polycarbonate, polybutylene terephthalat, polyethylene terephthalat, as well as polyurethane such as elastane and pellethane, as well as silicone, polyphosphazene, polyphenylene, polymer foams (of styrols and carbonates), polydioxanone, polyglycolide, polylactide such as poly-l-, poly-d-, and poly-d,l-lactide, as well as poly-ε-caprolacton, ethylvinyl acetate, polyethylene oxide, polyphosphoryl cholin, polyhydroxy butyric acids such as polyhydroxy valerate, as well as lipids such as cholesterol and cholesterol ester, as well as polysaccharides such as alginate, chitosan, levan, hyaluronic acid, uronide, heparin, dextran and cellulose, as well as proteins such as fibrin and albumin, as well as polypeptides and copolymers, blends and derivatives of these compounds.

3. The medical product according to claim 1, wherein the biostable and/or biodegradable polymer layer applied to the medical product has a thickness of 2 μm to 60 μm per layer.

4. The medical product according to claim 3, wherein the biostable and/or biodegradable polymer layer applied to the surface of the medical product has a thickness of 10 μm to 30 μm per layer.

5. The medical product according to claim 1, wherein in and/or on the biostable and/or biodegradable polymer layer there is at least one inhibitor of the transport protein P-glycoprotein in a concentration of 0.25-7.0 μg/mm².

6. The medical product according to claim 1, wherein the at least one pharmaceutically active substance is an antimicrobial, antimitotic, antimyotic, antineoplastic, antiphlogistic, antiproliferative, antithrombotic and/or vasodilatory active substance selected from the group consisting of triclosan, cephalosporin, aminoglycoside, nitrofurantoin, penicillins such as dicloxacillin, oxacillin as well as sulfonamide, metronidazol, 5-fluoruracil, cisplatin, vinblastin, vincristin, epothilones, endostatin, verapamil, statins such as cerivastatin, atorvastatin, simvastatin, fluvastatin, rosuvastatin as well as lovastatin, angiostatin, angiopeptin, taxanes such as paclitaxel, immuno suppressives or immuno modulators such as rapamycin or its derivatives such as bolimus, everolimus, deforloimus, novolimus, methotrexate, colchicin, flavopiridol, suramin, cyclosporin A, clotrimazol, flucytosin, griseofulvin, ketoconazol, miconazol, nystatin, terbinafin, steroids such as dexamethasone, prednisolone, corticosterone, budesonid, estrogen, hydrocortisone as well as mesalamine, sulfasalazin, heparin and its derivatives, urokinase, PPack, argatrobane, aspirin, abciximab, synthetic antithrombin, bivalirudin, enoxoparin, hirudin, r-hirudin, protamine, prourokinase, streptokinase, warfarin, flavonoids such as 7,3′,4′-trimethoxyflavon as well as dipyramidol, trapidil, nitroprusside, individually or in combination.

7. The medical product according to claim 6, wherein the at least one pharmaceutically active substance is selected from the group consisting of paclitaxel, rapamycin and its derivatives, atorvastatin, simvastatin, lovastatin and verapamil, individually or in combination.

8. The medical product according to claim 6, wherein the at least one pharmaceutically active substance is selected from the group consisting of paclitaxel and rapamycin, individually or in combination.

9. The medical product according to claim 1, wherein in and/or on the biostable and/or biodegradable polymer layer the at least one pharmaceutically active substance is present in a concentration of 0.2-3.5 μg/mm².

10. The medical product according to claim 1, wherein the medical product is a balloon catheter or a stent.

11. A method for the production of the medical product according to claim 1, comprising:
providing PSC833 as an inhibitor of the transport protein P-glycoprotein.

12. A method for the production of the medical product according to claim 1, comprising:
providing GF120918 as an inhibitor of the transport protein P-glycoprotein.

13. A method for the production of the medical product according to claim 1, comprising:
providing XR9576 as an inhibitor of the transport protein P-glycoprotein.

14. A method for the production of the medical product according to claim 1, comprising:
providing LY335979 as an inhibitor of the transport protein P-glycoprotein.

15. A method for the production of the medical product according to claim 1, comprising:
providing OC144-093 as an inhibitor of the transport protein P-glycoprotein.
